(19) United States
(12) Patent Application Publication
(10) Pub. No.: US 2026/0294380 A1
(43) Pub. Date: Oct. 1, 2026

(54) METHODS, SYSTEMS, AND APPARATUSES FOR IMAGE-GUIDED DELIVERY OF PROPAGATING WAVE ENERGY TO A TARGET

(71) Applicant: Softwave Tissue Regeneration Technologies, LLC, Alpharetta, GA (US)

(72) Inventor: John F. Warlick, Talking Rock, GA (US)

(73) Assignee: Softwave Tissue Regeneration Technologies, LLC, Alpharetta, GA (US)

(21) Appl. No.: 19/089,542

(22) Filed: Mar. 25, 2025

(51) Int. Cl. A61B 8/08; A61B 8/00; A61B 90/00; A61H 23/00; A61H 23/02

(52) U.S. Cl. CPC A61B 8/085; A61B 8/4263; A61B 8/5207; A61B 8/5223; A61H 23/008; A61H 23/0236; A61B 2090/3958; A61H 2201/5058

(57) ABSTRACT

Disclosed herein are methods, systems, and apparatuses enabling delivery of propagating wave energy to a target location in a system-aided manner. Such delivery of propagating wave energy to the target location in a system-aided manner may be performed in regard to a target location within a body of a patient to treat a wellness condition (i.e., medical condition, health condition, preventative treatment, elective treatment, etc.). Such methods, systems, and apparatuses provide an approach to using imaging of a target within a body that is to be treated with propagating wave energy for subsequent targeted delivery of such propagating wave energy to the target location.

### FIELD OF THE DISCLOSURE

The disclosures made herein relate generally to utilization of propagating wave energy for targeting and treatment purposes and, more particularly, to methods, systems, and apparatuses for treating a target location within a body via image-guided delivery of propagating wave energy.

### BACKGROUND

Shock Wave Therapy (SWT) is well known in the art in regard to operability and applicability. SWT involves delivery of acoustic shock waves (ASW) to a body for the purpose of providing therapeutic effect to portions of the body exposed to such acoustic shock waves. The acoustic shock waves are a form of pressure pulse (i.e., propagating wave energy) that acts on tissue, blood, and other cellular matter within the body for mitigating or eliminating undesirable health and medical conditions. A known benefit of SWT is the limited risk to healthy tissue, blood, and other cellular matter that may be directly or incidentally exposed to SWT treatment.

A particular type of SWT is Extracorporeal Shock Wave Therapy (ESWT). ESWT is a therapeutic modality of SWT involving the application of acoustic shock waves (i.e., energy thereof) at a surface of a body for treating pathologies and/or enhance beneficial physiological processes within the body. It is known that, as this treatment is increasingly applied to targets further from the body surface, the time and cost efficiencies of the treatment are challenged by the larger area in which treatment needs to be applied in order to ensure coverage while accounting for uncertainties in the target position. Additionally, the uncertainties in the target position as related to actual ASW trajectory has the potential to adversely impact treatment outcome, particularly for a target location that is relatively small in size as compared to the ASW at a depth within the body where the target location is located.

Therefore, methods, systems, and apparatuses enabling delivery of propagating wave energy to a target location within an enclosed space (e.g., within a body such as of a patient undergoing treatment for a wellness condition) in a system-aided manner would be advantageous, desirable and useful.

### SUMMARY OF THE DISCLOSURE

Embodiments of the disclosures made herein are directed to methods, systems, and apparatuses enabling delivery of propagating wave energy to a target location in a system-aided manner. In preferred embodiments, such delivery of propagating wave energy to a target location in a system-aided manner may be performed in regard to a target location within a body of a patient to treat a wellness condition (i.e., medical condition, health condition, preventative treatment, elective treatment, etc.). More specifically, embodiments of the disclosures made herein advantageously provide an approach to using imaging information associated with the target location for subsequent targeted delivery of propagating wave energy to the target location in a system-aided manner. Through such targeted delivery of propagating wave energy to the target location, efficacy treatment is enhanced due to accurate placement of the propagating wave energy relative to the target location whereby medical outcomes are improved and are achieved in a more efficient manner.

In one or more embodiments of the disclosures made herein, a system for treating a patient with acoustic shock waves comprises an ultrasound transducer, an acoustic shock wave applicator, and an information processing apparatus. The ultrasound transducer may be configured to output transducer spatial position information, to emit ultrasound energy for imaging a target location within the body, and to output imaging information generated by the imaging. The acoustic shock wave applicator may be configured to emit acoustic shock waves and to output applicator spatial position information. The information processing apparatus may be coupled to the ultrasound transducer and to the acoustic shock wave applicator for receiving the transducer spatial position information, the applicator spatial position information, and the imaging information respectively therefrom. The information processing apparatus may be configured for generating a mapping function using the transducer spatial position information. The mapping function correlates a plurality of transducer spatial positions derived from the transducer spatial position information to a respective portion of the imaging information. The respective portion of the imaging information was generated with the ultrasound transducer in a particular one of the transducer spatial positions.

In one or more embodiments of the disclosures made herein, an apparatus for treating a patient using propagating wave energy comprising a transducer, an applicator, and a feedback module operably coupled to the transducer and to the applicator. The transducer may be operable to emit a first configuration of propagating wave energy for enabling images of a target location within a body of the patient to be generated. Each of the images corresponds to a respective one of a plurality of transducer spatial positions relative to the target location. The applicator may be operable to emit a second configuration of propagating wave energy from a plurality of applicator spatial positions relative to the target location for treating a wellness condition at the target location. The feedback module correlates a current one of the applicator spatial positions to a particular one of the images when the current one of the applicator spatial positions is the same as or sufficiently similar to the respective one of the transducer spatial positions in which the transducer was positioned for enabling the particular one of the images to be generated.

In one or more embodiments of the disclosures made herein, a non-transitory computer-readable memory medium containing program instructions for controlling one or more data processors, when executed, to provide system-aided delivery of propagating wave energy for treating a wellness condition at a target location within a body of a patient. The method comprises receiving first spatial position information defining spatial positions of a first handheld device relative to the body. The first handheld device emits a first configuration of propagating wave energy. The method comprises receiving imaging information outputted by the first handheld device. The imaging information may be generated by the first handheld device using the propagating wave energy emitted thereby for imaging a target location within the body. The imaging may be performed with the first handheld device in a plurality of the spatial positions thereof. The method comprises receiving second spatial position information defining spatial positions of a second handheld device relative to the body. The second handheld device emits a second configuration of propagating wave energy selected for treating a wellness condition at the target location. The method comprises generating an image of the target location corresponding to a particular one of the spatial positions of the second handheld device. The generating may be performed as a function of the first spatial position information, the second spatial position information, and the imaging information.

In one or more embodiments, the ultrasound transducer and the acoustic shock wave applicator each comprise a first portion of a spatial position sensing apparatus that respectively outputs a signal representative of said spatial position information.

In one or more embodiments, a second portion of the spatial position sensing apparatus may be one of coupled to and integral with the information processing apparatus, wherein the second portion of the spatial position sensing apparatus receives said spatial position information and determines therefrom a relative spatial position with respect to a fixed reference position.

In one or more embodiments, the first portion of the spatial position sensing apparatus includes a spatial field generator (e.g., a magnet spatial field generator) and the second portion of the spatial position sensing apparatus includes a spatial field sensor.

In one or more embodiments, a spatial referencing module may be one of coupled to and integral with the information processing apparatus and the spatial referencing module receives the spatial position information from the ultrasound transducer and from the acoustic shock wave applicator and determines therefrom a respective spatial position thereof with respect to a fixed reference position.

In one or more embodiments, the respective portion of the imaging information enables an image of the target location to be rendered therefrom.

In one or more embodiments, the information processing apparatus may be further configured for determining, as a function of the mapping function and a particular one of a plurality of applicator spatial positions, the respective portion of said imaging information generated with the ultrasound transducer in the particular one of said transducer spatial positions that is same or sufficiently the same as the particular one of said applicator spatial positions thereby enabling real-time spatial position of the acoustic shock wave applicator to be adjusted based at least partially upon said imaging information.

In one or more embodiments, the information processing apparatus may be further configured for deriving each of said applicator spatial positions from said applicator spatial position information.

In one or more embodiments, the information processing apparatus may be further configured for at least one of: causing display of the respective portion of said imaging information in real-time as a function of said applicator spatial position information; and providing confirmation that an energy wave emissions axis of the acoustic shock wave applicator is colinearly aligned with or sufficiently colinearly aligned with an energy wave emissions axis of the ultrasound transducer when the respective portion of said imaging information was generated.

In one or more embodiments, being in the particular one of said transducer spatial positions that is same or sufficiently the same as the particular one of said applicator spatial positions includes an energy wave emissions axis of the acoustic shock wave applicator being colinearly aligned with or sufficiently colinearly aligned with an energy wave emissions axis of the ultrasound transducer when the respective portion of said imaging information was generated.

In one or more embodiments, the first configuration of propagating wave energy may be ultrasound energy and the second configuration of propagating wave energy may be acoustic shock wave energy.

In one or more embodiments, the first configuration of propagating wave energy may be selected for enabling imaging of the target location and the second configuration of propagating wave energy may be selected for treating the wellness condition.

In one or more embodiments, a longitudinal reference axis of a spatial field generator is aligned with an energy emissions axis of the respective one of the transducer and the applicator attached thereto.

In one or more embodiments, the current one of said applicator spatial positions being sufficiently similar to the respective one of said transducer spatial positions includes at least one of: an energy emissions axis of the applicator when in the current one of said applicator spatial positions being within a prescribed lateral offset from an energy emissions axis of the transducer when in the respective one of said transducer spatial positions and an energy emissions axis of the applicator when in the current one of said applicator spatial positions being within a prescribed angular offset from an energy emissions axis of the transducer when in the respective one of said transducer spatial positions.

In one or more embodiments, the first spatial position information and the second spatial position information respectively include information characterizing an orthogonal position of a spatial field generator of the respective one of the handheld devices relative to a reference location and information characterizing angular rotation of the spatial field generator relative to a reference axis.

In one or more embodiments, generating the image of the target location corresponding to the particular one of said spatial positions of the second handheld device includes: accessing a mapping function correlating a plurality of first handheld device spatial positions derived from said first handheld device spatial position information to a respective portion of said imaging information and wherein the respective portion of the imaging information was generated with the first handheld device in a particular one of said transducer spatial positions; and determining, as a function of the mapping function and the particular one of said spatial positions of the second handheld device, the respective portion of said imaging information generated with the first handheld device in the particular one of said first handheld device spatial positions that is same or sufficiently the same as the particular one of said spatial positions of the second handheld device.

These and other objects, embodiments, advantages and/or distinctions of the present invention will become readily apparent upon further review of the following specification, associated drawings and appended claims.

### DEFINITIONS AND ASSOCIATED DISCLOSED FUNCTIONALITIES

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plane waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to one or more embodiments of the disclosures made herein is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nanoseconds (ns) up to some milliseconds (ms). Very fast pressure pulses are called shock waves. Shock waves used in medical applications do have amplitudes above 0.1 MPa and rise times of the amplitude can be below 1000 ns, preferably at or below 100 ns.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

“Telomeres” play a central role in cell fate and aging by adjusting the cellular response to stress and growth stimulation on the basis of previous cell divisions and DNA damage. At least a few hundred nucleotides of telomere repeats must “cap” each chromosome end to avoid activation of DNA repair pathways. Repair of critically short or

“uncapped” telomeres by telomerase or recombination is limited in most somatic cells and apoptosis or cellular senescence is triggered when too many “uncapped” telomeres accumulate. Telomerase and recombinations thereof are examples of proteins that may be released in response to stimulation of cells in accordance with embodiments of the disclosures made herein that promote regulation and lengthening of telomeres such as, for example, via activation of DNA repair pathways.

Waves/wave fronts described as being “focused” or “having focusing characteristics” means in the context of the present invention that the respective waves or wave fronts are traveling and increase their amplitude in direction of the focal point. Per definition, the energy of the wave will be at a maximum in the focal point or, if there is a focal shift in this point, the energy is at a maximum near the geometrical focal point. Both the maximum energy and the maximal pressure amplitude may be used to define the focal point.

“Standard of Care”: Treatment that is accepted by medical experts as a proper treatment for a certain type of medical disorder (e.g., a disease) and that is widely used by health care professionals.

“Extracorporeal” occurring or based outside the living body.

### BRIEF DESCRIPTION OF THE DRAWINGS

FIG. 1A is a diagrammatic view of a propagating wave energy system in accordance with one or more embodiments of the disclosures made herein, wherein the system is in an imaging mode of operation.

FIG. 1B is a diagrammatic view of the propagating wave energy system of FIG. 1A, wherein the system is in a treatment mode of operation prior to target location alignment.

FIG. 1C is a diagrammatic view of a propagating wave energy system of FIG. 1A, wherein the system is in a treatment mode of operation during target location alignment.

FIG. 1D is a diagrammatic view of a process for system-aided applicator alignment in accordance with one or more embodiments of the disclosures made herein with a desired spatial treatment position STP, wherein the process is in a state of emissions axis mis-alignment relative to a designed spatial treatment position.

FIG. 1E is a diagrammatic view of a process for system-aided applicator alignment in accordance with one or more embodiments of the disclosures made herein with a desired spatial treatment position STP, wherein the process is in a state of emissions axis alignment relative to a designed spatial treatment position.

FIG. 2 is a flow diagram showing operations for performing delivery of propagating wave energy to a target location in a system-aided manner in accordance with one or more embodiments of the disclosures made herein.

FIG. 3 is a flow diagram showing operations for enabling delivery of propagating wave energy to a target location in a system-aided manner in accordance with one or more embodiments of the disclosures made herein.

FIG. 4 is a block diagram of a system configured for providing delivery of propagating wave energy to a target location in a system-aided manner in accordance with one or more embodiments of the disclosures made herein.

FIG. 5 is a diagrammatic view of a mapping of propagating wave energy delivery relative to delivery location in accordance one or more embodiments of the disclosures made herein.

FIG. 6 is a block diagram of a network infrastructure including a computing system adapted for implementing delivery of propagating wave energy to a target location in a system-aided manner in accordance with one or more embodiments of the disclosures made herein.

FIG. 7 is a diagrammatic view of a pressure pulse/shock wave (PP/SW) generator showing focusing characteristics of transmitted acoustic pressure pulses.

FIG. 8 is a diagrammatic view of a pressure pulse/shock wave generator with plane wave characteristics.

FIG. 9 is a diagrammatic view of a pressure pulse shock wave generator (i.e., a shock wave head of a propagating energy wave source) with divergent wave characteristics.

FIG. 10 is a diagrammatic view of an exemplary acoustic shock wave apparatus in accordance with one or more embodiments of the disclosures made herein.

### DETAILED DESCRIPTION

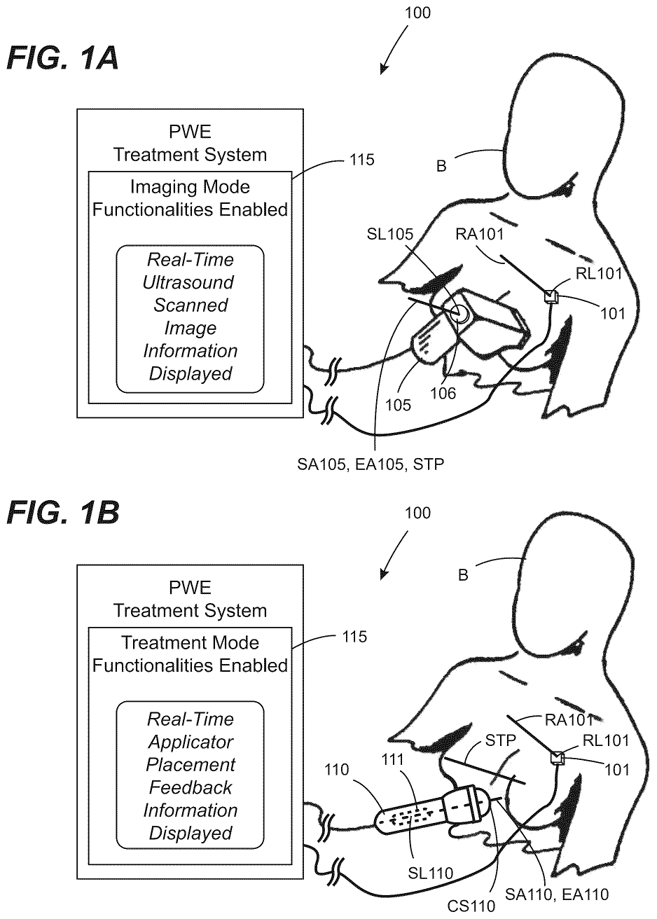

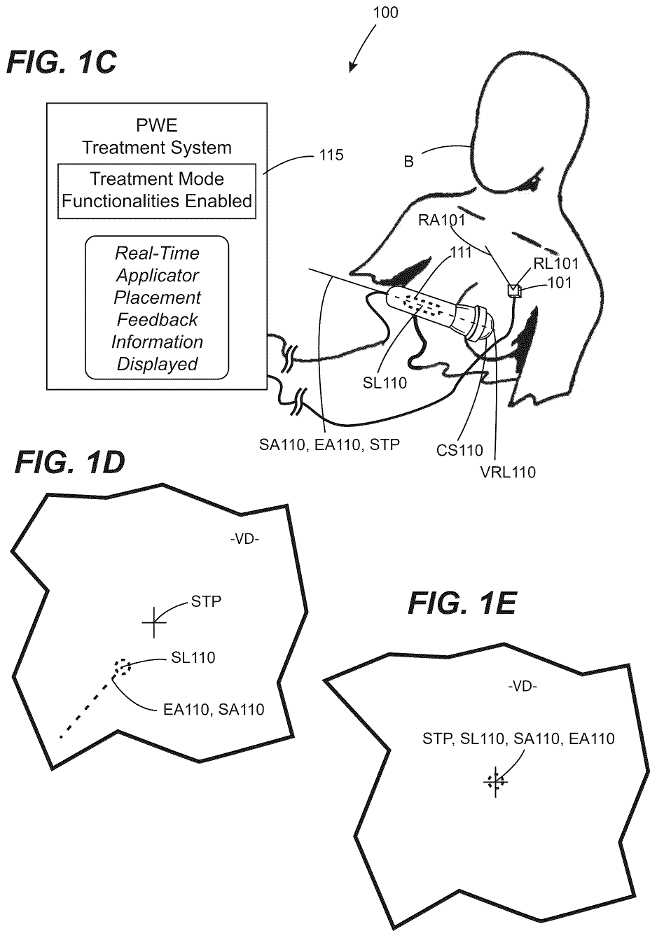

Referring to FIG. 1A, a propagating wave energy system in accordance with one or more embodiments of the disclosures made herein is shown—i.e., system 100. The system 100 provides for delivery of propagating wave energy (PWE) to a target location within a body B (i.e., the treatment target) of a person patient who may be a patient or otherwise that is subject to a treatment for the wellness condition (or otherwise) in a system-aided manner. The wellness condition may be associated with a medical condition, a health condition, a preventative treatment, an elective treatment, or the like that may reside at, near, or activated by (e.g., via reflexology) the target location.

Advantageously and beneficially, the system 100 provides an approach to using imaging information associated with the target location for subsequent targeted delivery of propagating wave energy to the target location in a system-aided manner. Through such system-aided (i.e., targeted) delivery of propagating wave energy to the target location, efficacy of treatment is enhanced due to accurate placement of the propagating wave energy relative to the target location whereby medical outcomes are improved and are achieved in a more efficient manner.

As shown in FIGS. 1A and 1B, the system 100 is depicted as an apparatus that comprises a transducer 105, an applicator 110, and a feedback module 115. The feedback module 115 is operably coupled to the transducer 105 and to the applicator 110. The transducer 105 is used for imaging a treatment location using a first configuration of propagating wave energy. The applicator 110 is used for treating the treatment location with a second configuration of propagating wave energy. The first and second configurations of propagating wave energy may be the same or different than each other.

The transducer 105 is operable to emit a first configuration of propagating wave energy for enabling images of a target location within a body of the patient to be generated. For example, the first configuration of propagating wave energy may be ultrasound wave energy or other energy suitable for internal bodily imaging. Ultrasound is a preferred and well known technique for imaging in accordance with embodiments of the disclosures made herein. Similarly, ultrasound imaging system construction, operability, and functionality is well known. Specific details of ultrasound imaging systems and methodologies as related to imaging within a body of a patient are disclosed in U.S. Pat. and/or Patent Application Publication Nos. 6,540,682; 6,135,961; 5,839,442; and 5,709,209, which are incorporated herein in their entirety by reference.

The applicator 110 is operable to emit a second configuration of propagating wave energy for treating the wellness condition at the target location. For example, the second configuration of propagating wave energy may be acoustic shock wave energy or other suitable type of asymmetric pressure pulse energy. Acoustic shock waves are a preferred and well known form of propagating wave energy for treating wellness conditions in accordance with embodiments of the disclosures made herein. Similarly, acoustic shock wave system construction, operability, and functionality is well known. Specific details of acoustic shock wave imaging systems and methodologies as related to treating wellness conditions within a living person, patient or otherwise) are disclosed in U.S. Pat. and/or Patent Application Publication Nos. 7,470,240; 7,507,213; 7,988,648; and 20210322664, which are incorporated herein in their entirety by reference.

The transducer 105 and the applicator 110 each include a respective spatial field generator 106, 111 that outputs output respective spatial position information. In some embodiments, the spatial field generators 106, 111 may be magnet spatial field generator. However, the spatial field generators 106, 111 are not limited to a particular type or configuration.

The spatial position information is that which enables a spatial position of the spatial field generators 106, 111 to be respectively determined by the system 100 such as in response to respective movement of the transducer 105 and the applicator 110 relative to a spatial field sensor 101 of the system 100. To this end, for a respective one of the spatial field generators 106, 111, the spatial position information may define or otherwise characterize its rotational orientation (e.g., about local orthogonal x-y-z directions). An intersection of the orthogonal axes defines an instant (i.e., local) position in space. For example, as shown in FIG. 1A, the spatial field generator 106 of the transducer 105 defines a respective spatial location SL105 and a spatial axis SA105 extending through the spatial location SL105 of the transducer 105. The spatial axis SA105 may extend colinearly with an emissions axis EA105 of the spatial field generator 106 of the transducer 105. Similarly, the spatial field generator 111 of the applicator 110 defines a respective spatial location SL110 and a spatial axis SA110 extending through the spatial location SL110 of the applicator 110. The spatial axis SA110 may extend colinearly with an emissions axis EA110 of the spatial field generator 111 of the applicator 110. Thus, relative to the respective spatial location, the respective spatial rotation is known for both transducer 105 and the applicator 110.

The spatial field sensor 101 may be affixed to the body B, such as near the target location. Accordingly, the spatial field sensor 101 may be located at a fixed position relative to the body B, the transducer 105, and the applicator 110. The spatial field sensor 101 defines a reference spatial location RL101 and a reference spatial axis RA101.

As is well known in the art, the spatial position and spatial rotation of a spatial field generator may be specified relative to the fixed position (i.e., spatial location and spatial axis) of a spatial field sensor operable to sense the spatial position information being outputted from a spatial field generator. For example, where a spatial field generator emits a magnetic field, the spatial position and spatial rotation of the spatial field generator may be specified relative to the fixed position of a spatial field sensor operable to sense the magnetic field. In the case of the spatial field sensor 101, the system 100 preferably is operable for (e.g., via an information processing apparatus) determining and specifying the spatial position and spatial rotation of the spatial field generators 106, 111 relative to the fixed position (i.e., spatial location and spatial axis) of the spatial field sensor 101. Such specifying may be in the form of a position of a respective one of the spatial field generators 106, 111 relative to orthogonal axes including the reference spatial axis RA101 and a spatial position relative to the orthogonal axes (e.g., angular rotation about the respective one of the spatial locations SL105, SL110). In these regards, relative spatial locations and spatial axes of the spatial field generator 106 of the transducer 105 and relative spatial locations and spatial axes of the spatial field generator 111 of the applicator 110 (i.e., relative to a known, fixed position) may be determined and specified to enable spatial tracking of the transducer 105 and the applicator 110.

Specific details of spatial position generator/sensor systems and methodologies thereof enabling spatial tracking of objects are disclosed in U.S. Pat. and/or Patent Application Publication Nos. 3,983,474; 4,054,881; 4,622,644; 4,742,356; 5,558,091; 5,913,820; 6,073,043; and 6,774,624, which are incorporated herein in their entirety by reference.

Known geometry and/or dimension of the transducer 105 and the applicator 110 will allow the relative spatial locations and spatial axes of the respective spatial field generator 106, 111 to be virtually transposed to a given location of the transducer 105 and/or the applicator 110, respectively (i.e., the virtual spatial reference location and the virtual spatial reference axis associated with a respective one of the spatial field generator 106, 111). The virtual spatial reference location and the virtual spatial reference axis may be at a surface of the transducer that engages the surface of the body on an energy emissions axis EA105 of the transducer 105 (i.e., axis along which propagating waves are emitted from the transducer 105). Similarly, the virtual spatial reference location and the virtual spatial reference axis may be at a surface of the applicator that engages the surface of the body on an energy emissions axis EA110 of the applicator 110 (i.e., axis along which propagating waves are emitted from the applicator 110).

Referring to FIG. 1A, during use of the system 100 in an imaging mode of operation, the transducer 105 is used for imaging (i.e., via the first configuration of propagating wave energy) a target location within the body B of a person who may be a patient or otherwise that is subject to a treatment for the wellness condition (or otherwise). Such imaging is well known to include moving the transducer over a skin surface of the body B while viewing a real-time display of images of the target location generated using imaging information outputted by the transducer 105. Such images may be in the form of a real-time streaming video image derived from imaging information generated by the transducer 105.

Spatial positions of the transducer 105 may be denoted during imaging. For example, with the transducer 105 at a desired spatial treatment position STP, a user of the system (e.g., the transducer operator) may selectively log in a record one or more desired spatial positions of the transducer 105 at which subsequent treatment may be made via the applicator 110. For example, selective marking may record notation of the one or more desired spatial positions, record notation of one or more timestamps associated with a given portion of the imaging information, or the like for enabling retrieval of one or more given points of time during the imaging mode.

With imaging completed, the system 100 is transitioned to a treatment mode of operation in which the objective is to deliver a second configuration of propagating wave energy to the target location in a system-aided manner. In some embodiments, the second configuration of propagating wave energy may be the same or sufficiently the same as the second configuration of propagating wave energy. For example, both configurations of propagating wave energy may be of the ultrasound type. The second configuration of propagating wave energy is sufficiently suited for treatment of the wellness condition at, near, or activated by the target location. Acoustic shock wave energy is a preferred configuration of propagating wave energy for treating wellness conditions.

Referring to FIG. 1B, in the treatment mode of operation, the applicator 110 is again moved over the skin surface of the body B, while assessing real-time applicator placement feedback information. In preferred embodiments, such assessing of the real-time applicator placement feedback information may include viewing displayed images of the target location generated during the imaging mode of operation and correlated to spatial positions of the applicator 110 as a function of spatial positions of the transducer 105. Such images may be in the form of a real-time streaming video image derived from the imaging information generated during the imaging mode of operation. In some embodiments, such assessing of the real-time applicator placement feedback information includes viewing information confirming that a current spatial placement of the applicator 110 utilizing spatial tracking and imaging information generation functionalities disclosed in accordance with embodiments of the disclosures made herein.

Real-time applicator placement feedback information correlates each of a plurality of target location images and a position in which the transducer 105 was correspondingly positioned spatially to respectively create each of such images to a current position of the applicator 110 during the treatment mode of operation. Accordingly, spatial tracking and imaging information generation functionalities disclosed in accordance with embodiments of the disclosures made herein advantageously enable system-aided delivery of the second configuration of propagating wave energy to the target location. Functionalities provided for during the imaging mode of operation and the treatment mode of operation (e.g., the spatial tracking and imaging information generation functionalities) may be provided for by a feedback module of the system 100 which may be implemented by structure and operability of an information processing apparatus of the system 100.

Referring to FIG. 1C, when the applicator 110 is in a desired spatial treatment position STP for providing treatment of the wellness condition at the target location, propagating wave energy of the second configuration may be delivered via the applicator 110. As shown, with the applicator 110 in the desired spatial treatment position STP, the energy emissions axis EA110 of the applicator extends colinearly with or sufficiently colinearly aligned with the desired spatial treatment position STP. Confirmation of axis alignment may be made by any suitable means such as, for example, target location imaging information (e.g., via real-time display of spatial position-correlated images derived from imaging information generated during imaging mode), visual acknowledgement of axis alignment, textual means acknowledgement of axis alignment, audible acknowledgement of axis alignment, tactile acknowledgement of axis alignment, and/or the like.

Being sufficiently colinearly aligned includes an acceptable amount of mis-alignment between the energy emissions axis EA110 of the applicator 110 and the desired spatial treatment position STP. The misalignment may be angular mis-alignment, orthogonal mis-alignment, or both. The acceptable amount is dependent upon factors including, but not limited to waveform, of the propagating wave energy, size of the target location, position of the target location, power level of the propagating wave energy and the like. For example for a smaller target location located mid-mass or deeper in the body, a higher resolution of alignment may be required. When a convergent waveform is selected rather than a divergent waveform, a higher resolution of alignment may be required. An objective of axis alignment may be to ensure that not less than a desired amount of propagating wave energy impinges upon the target location (e.g., not less than 95% of a theoretical maximum (e.g., assuming full colinear alignment), not less than 80% of theoretical maximum, not less than 70% of theoretical maximum, etc.) Alignment may also be limited to practicalities of achieving full colinear alignment (e.g., expectation of achieving only within 95%, 80,%, or 70% alignment relative to full colinear alignment).

Referring to FIGS. 1D and 1E, an embodiment of a process for system-aided alignment of the applicator 110 with a desired spatial treatment position STP is disclosed. Advantageously, in this process, the system generates a 2-dimensional representation of the 3D topography of a surface over which the imaging device 105 and the applicator 110 are translated during their respective use which provides for a simplified approach for achieving alignment of the energy emissions axis EA110 of the applicator 110 and the desired spatial treatment position STP. Visualization of images of the target location is replaced by visualization of alignment targeting components that enable a user to bring a spatial position of the applicator 110 into alignment with a desired spatial treatment position. This process for system-aided alignment may be carried out via one or more functional components of the system 100—e.g., an information processing apparatus, a feedback module, a treatment energy delivery module, etc.

The spatial treatment position STP has been denoted during imaging of the target location such that it can be retrieved for subsequent utilization during treatment of the target location. On a visual display VD of the system 100, the relative positions of the energy emissions axis EA110 of the applicator 110 and the desired spatial treatment position STP are displayed. In FIG. 1D, there is spatial separation (axial and lateral) between the energy emissions axis EA110 of the applicator 110 and the desired spatial treatment position STP. In FIG. 1E, the applicator 110 has been manipulated (i.e., manually or otherwise) such that the energy emissions axis EA110 of the applicator 110 and the desired spatial treatment position STP are aligned—e.g., axes colinearly aligned or sufficiently colinearly aligned. Image information (e.g., video or images) may be displayed in combination with the energy emissions axis EA110 of the applicator 110 and the desired spatial treatment position STP (e.g., the emissions axis EA110 of the applicator 110 and the desired spatial treatment position STP displayed ton top of the image information). Also, in some embodiments, the energy emissions axis EA110 of the applicator 110 and/or the desired spatial treatment position STP may be omitted from being displayed).

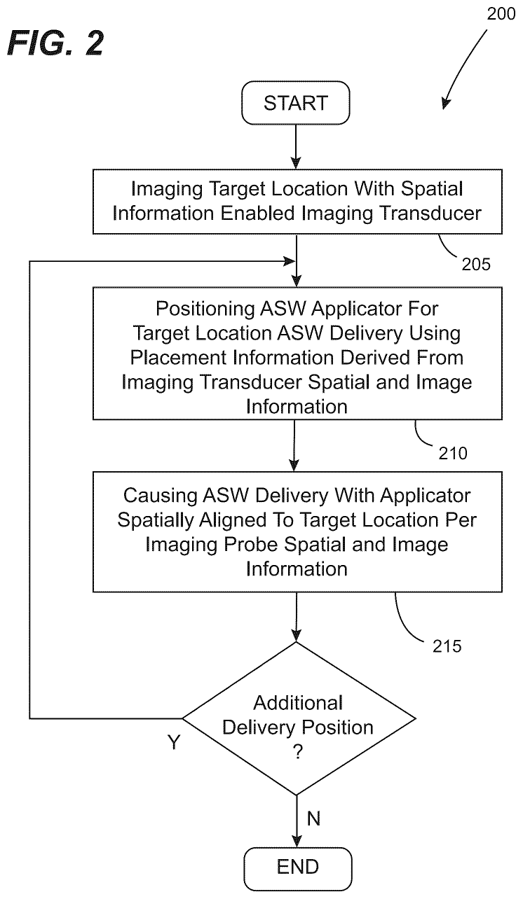

FIG. 2 shows a flow diagram depicting a method 200 of user steps for carrying out system-aided (i.e., targeted) delivery of propagating wave energy to a target location in accordance with one or more embodiments of the disclosures made herein. The method 200 is discussed in reference to system components discussed above in reference to the system 100 in FIGS. 1A-1C. The method 200 provides for system-aided delivery of propagating wave energy utilized for treatment of a wellness condition. Such system-aided delivery includes real-time applicator placement feedback information (e.g., a mapping function) that correlates each of a plurality of target location images and a position in which the transducer 105 was spatially corresponding positioned to respectively create each of such images to a current position of the applicator 110 when delivering propagating wave energy for treatment of the wellness condition.

A user step 205 is performed for imaging a target location with an imaging transducer (e.g., transducer 105) that enables determination and recordation of spatial position information of the transducer in combination with generation of imaging information for the target location. The transducer performs the imaging through use of propagating wave energy such as, for example, ultrasound energy—i.e., a first configuration of propagating wave energy. As discussed above, such spatial position information characterizes a real-time spatial position (e.g., orthogonal and angular) of the transducer 105 during imaging of the target location. Imaging a target location generates imaging information from which images can be generated (i.e., derived). In preferred embodiments, imaging is performed using ultrasound. In preferred embodiments, imaging information enables a real-time video playback of images (e.g., video playback). The imaging information and spatial position information are recorded or otherwise stored in a correlated manner such that a position of the transducer when a particular portion of the imaging information was generated is known.

A user step 210 is performed for positioning a propagating wave energy applicator (e.g., applicator 110) for target location propagating wave energy delivery using placement information derived from the imaging information and spatial position information. The placement information may be a mapping function and correlates a current one of the applicator spatial positions to a particular one of the images. The correlation is made when the current one of the applicator spatial positions is the same as or sufficiently similar to the respective one of the transducer spatial positions in which the transducer was positioned for enabling the particular one of the images to be generated. The current one of the applicator spatial positions is one of a plurality of applicator spatial positions that the applicator moves through to acquire a desired spatial treatment position (STP). As discussed above, there may be a plurality of spatial treatment positions for a given target location. Each spatial treatment position preferably corresponds to a spatial position achieved during imaging of the target location.

After a desired spatial treatment position is acquired, a user step 215 is performed for causing propagating wave energy to be delivered to the target location for treating the wellness condition. The applicator performs the treatment by emitting a propagating wave energy such as, for example, acoustic shock wave energy—i.e., a second configuration of propagating wave energy in the form of acoustic shock waves. Causing the delivery of the propagating wave energy is performed while an energy emissions axis of the applicator is colinearly aligned with or sufficiently colinearly aligned with the desired spatial treatment position. Alignment of the applicator with the desired spatial treatment position may be facilitated via display of a respective portion of the imaging information in real-time as a function of the applicator spatial position as it is moved for acquiring the desired spatial treatment position. Alignment of the applicator with the desired spatial treatment position may also be facilitated via providing confirmation that the energy wave emissions axis of the applicator is colinearly aligned with or sufficiently colinearly aligned with an axis defining the desired spatial treatment position (i.e., the energy wave emissions axis of the transducer when the respective portion of said imaging information was generated.) The user steps for positioning the applicator and causing delivery of the treatment propagating wave energy may be repeated for each desired spatial treatment position from which the target location is to be treated.

A propagating energy wave source delivering propagating wave energy in accordance with embodiments of the disclosures made herein may be any type of source of propagating energy waves that offers propagating energy waves in a form suitable for facilitating wellness condition treatment in accordance with embodiments of the disclosures made herein. Although acoustic shock waves are a suitable type of propagating energy wave (i.e., pressure pulses) useful for wellness condition treatment in accordance with embodiments of the disclosures made herein, a skilled person will understand that other types of propagating energy waves (e.g., radial pressure waves, acoustic pneumatic waves, ultrasonic waves, and the like) may be useful for wellness condition treatment in accordance with embodiments of the disclosures made herein.

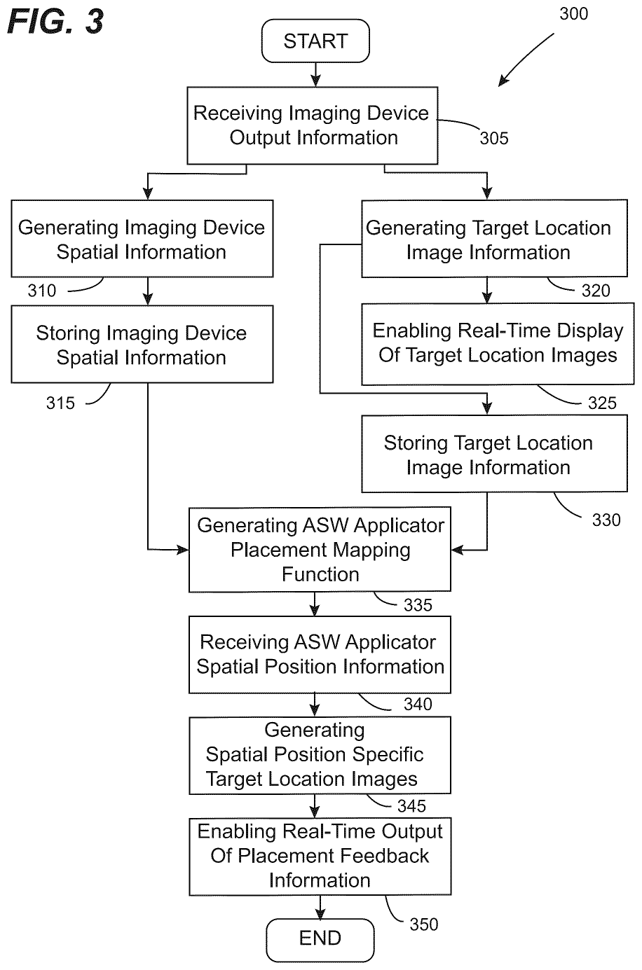

Referring now to FIG. 3, a method 300 of operational steps for enabling system-aided (i.e., targeted) delivery of propagating wave energy to a target location in accordance with one or more embodiments of the disclosures made herein. In some embodiments, an information processing apparatus may perform the operational steps of the method 300. The operational steps of the method 300 may define a feedback module of a system in accordance with one or more embodiments of the disclosures made herein and may be defined by program instructions. The information processing apparatus may be embodied by one or more data processors coupled to non-transitory computer-readable memory medium containing program instructions for controlling the one or more data processors, where execution of the program instructions cause system-aided delivery of propagating wave energy for treating a wellness condition at a target location within a body of a patient (e.g., those defining the method 100). Such system-aided delivery includes real-time propagating wave energy applicator placement feedback information (e.g., a mapping function) that correlates each of a plurality of target location images and a position in which an imaging transducer was correspondingly positioned spatially to respectively create each of such images to a current position of the applicator when delivering propagating wave energy for treatment of the wellness condition.

An operational step 305 may be performed for receiving output information generated by an imaging device during imaging of a target location within a body. The imaging device may be an ultrasound transducer and may be a first handheld device of a system in accordance with embodiment of the disclosures made herein. The output information includes spatial position information and imaging information. The output information may be embodied as one or more signals. Images of a target location that has been imaged with the imaging device may be derived from a first signal or respective portion (e.g., first portion) of a unitary signal (i.e., include spatial position information and imaging information) and spatial positions of the imaging device may be derived from a second signal or respective portion (e.g., second portion) of the unitary signal.

An operational step 310 may be performed for generating spatial position information, followed by an operation 315 of storing the spatial position information (e.g., in a digital format as data). As discussed above, the spatial position information may be generated by a spatial field generator and defines a position of the imaging device in free space or relative to a fixed reference location. The spatial position information may include correlation enabling information (e.g., timestamp information) for enabling correlation of the spatial position information to other information. The spatial position of the imaging device may include information characterizing an angular position of the imaging device (e.g., about orthogonal axes) and/or may include information characterizing an orthogonal position of the imaging device (i.e., relative to orthogonal axes).

An operational step 320 may be performed for generating imaging information for the target location, followed by an operational step 325 of enabling real-time display of images of the target location and an operational step 330 for storing the imaging information (e.g., in a digital format as data). In preferred embodiments, enabling real-time display of images may include enabling display of the images during imaging of the target location and may include denoting a particular portion of the imaging information with a particular spatial position of the imaging device at which the particular portion of the imaging information was generated. The imaging information may include correlation enabling information (e.g., timestamp information) for enabling correlation of the imaging information to other information. The real-time display of images may include display of video (e.g., streaming images).

An operational step 335 may be performed for generating a correlation between one or more imaging device spatial positions derived from the spatial position information of the imaging device to a respective portion of the imaging information. The correlation may be in the form of a mapping function (e.g., formulaic relationship between imaging device spatial position and portion of the image information) or a mapping table (e.g.,. 1:1 mapping of imaging device spatial position to a portion of the image information). The respective portion of the imaging information is generated with the imaging device in a particular one of imaging device spatial positions. Such correlation enables image information to be outputted as a function of a particular spatial position of a plurality of spatial positions of the propagating wave energy applicator (e.g., acoustic shock wave applicator) based upon a corresponding spatial position of the imaging device.

Through such correlation, image information (e.g., images derived therefrom) may be used to aid in placement of the propagating wave energy applicator at a desired spatial treatment position when a current spatial position of the propagating wave energy applicator is the same as or sufficiently similar to a desired spatial treatment position (i.e., a particular spatial position of the imaging device at which a respective portion of the imaging information was generated). For example, the current propagating wave energy applicator spatial position being sufficiently similar to the respective one of the imaging device spatial positions may include an energy emissions axis of the propagating wave energy applicator when in the current one of the propagating wave energy applicator spatial positions being within a prescribed lateral offset from an energy emissions axis of the imaging device when in the respective one of the imaging device spatial positions (i.e., the desired spatial treatment position) and/or an energy emissions axis of the propagating wave energy applicator when in the current one of the propagating wave energy applicator spatial positions being within a prescribed angular offset from the energy emissions axis of the imaging device applicator when in the respective one of the imaging device spatial positions.

An operational step 340 may be performed for receiving spatial position information for the propagating wave energy applicator. The spatial position information for the propagating wave energy applicator is received in response to the propagating wave energy applicator being moved relative to the target location for acquiring a desired spatial treatment position at which propagating wave energy will be delivered for treating a wellness condition at the target location. The spatial position information for the propagating wave energy applicator may be embodied as one or more signals.

An operational step 345 may be performed for generating images of the target location that correspond to a spatial position of the propagating wave energy applicator for enabling system-aided delivery of propagating wave energy at the target location. In preferred embodiments, the images of the target location that correspond to a current spatial position of the propagating wave energy applicator are derived as a function of the current spatial position of the propagating wave energy applicator in accordance with the correlation between the one or more imaging device spatial positions derived from the spatial position information of the imaging device to a respective portion of the imaging information, as discussed above in reference to operational step 335.

An operational step 350 is performed for enabling real-time output of placement feedback information for aiding in placement of the propagating wave energy applicator at a desired spatial treatment position. As the propagating wave energy applicator is moved relative to the target location, placement information is outputted. The placement information is configured to and serves to enable a user of the propagating wave energy applicator to efficiently move the propagating wave energy applicator toward and into effective alignment with a desired spatial treatment position.

In some embodiments, enabling real-time output of the placement feedback information may include causing display of the respective portion of the imaging information that is correlated to a current spatial position of the propagating wave energy applicator. The real-time display of images may include display of video (e.g., streaming images). In some embodiments, enabling real-time output of the placement feedback information may include providing confirmation that an energy wave emissions axis of the propagating wave energy applicator is colinearly aligned with or sufficiently colinearly aligned with an energy wave emissions axis of the ultrasound transducer when the respective portion of the imaging information was generated during imaging of the target location. Such confirmation may be in any number of forms including, but not limited to, visual acknowledgement of axis alignment, textual means acknowledgement of axis alignment, audible acknowledgement of axis alignment, tactile acknowledgement of axis alignment, and/or the like.

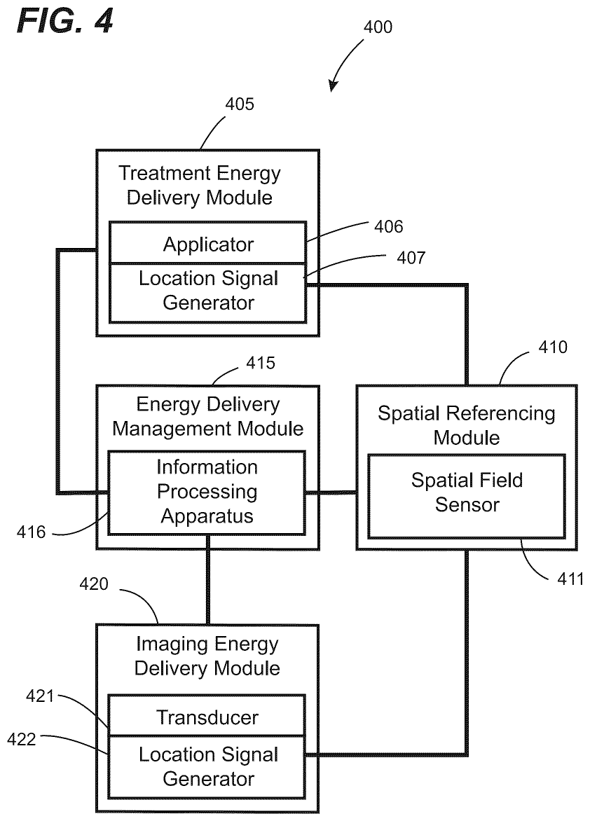

Referring to FIG. 4, a system in accordance with one or more embodiments of the disclosures made herein is shown—i.e., the system 400. The system 400 is operable for enabling modification of a biome biological profile of a biome region of a patient in accordance with one or more embodiments of the disclosures made herein. For example, the system 400 may be configured in a manner supporting execution of the methodologies 100, 200, 300 discussed above in reference to FIGS. 1A-1C, 2, and 3.

The system 400 includes a treatment energy delivery module 405, a spatial referencing module 410, an energy delivery management module 415, and an imaging module 420. The treatment energy delivery module 405 is operably coupled to the spatial referencing module 410 and the energy delivery management module 415. The energy delivery management module 415 is operably coupled to the imaging module 420. The spatial referencing module 410 is operably coupled to the treatment energy delivery module 405, the imaging module 420, and the energy delivery management module 415. All or some of the modules 405, 410, 415, 420 may be standalone systems. Two or more of the modules 405, 410, 415, 420 may be integral with each other as a standalone system, apparatus, or device. Embodiments of the disclosures made herein are not limited to a particular physical structure, but rather are characterized by structure and/or functionality as disclosed herein and appreciated in view of the disclosures made herein.

The treatment energy delivery module 405 includes an applicator 406 and a first location signal generator 407. The applicator 406 is a device from which propagating energy waves are emitted for enabling treatment of a respective portion of a body (i.e., target location at a spatial treatment position). The first location signal generator 407 is coupled to (e.g., fixedly attached to) the applicator 406 for outputting a signal that enables relative position and/or motion of the applicator 406 to be tracked and/or recorded. Specific details of acoustic shock wave delivery (i.e., propagating wave energy) systems and treatment therewith are disclosed in U.S. Pat. and/or Patent Application Publication Nos. 7,470,240; 7,507,213; 7,988,648; and 20210322664, which are incorporated herein in their entirety by reference.

The spatial referencing module 410 may be operably coupled (e.g., wirelessly) to the first location signal generator 407 that is coupled (e.g., physically) to or integral with the applicator 406 of the treatment energy delivery module 405 to receive or sense a spatial position signal outputted by the first location signal generator 407 of the treatment energy delivery module 405. In this respect, the first location signal generator 407 may be a first sensor of the spatial referencing module 410 that serves to enable the position of the applicator 406 to be defined (i.e., a first position-defining device). The spatial referencing module 410 may be operably coupled to the treatment energy delivery module 405 in a plurality of ways, including, but not limited to: unitarily embodied with the propagating energy wave module 405, integral with the treatment energy delivery module 405, a discrete unit operably connected to the treatment energy delivery module 405, or operably coupled to the treatment energy delivery module 405 but integral with/coupled to the energy delivery management module 415.

The spatial referencing module 410 is operable to utilize the spatial position signal outputted by the first location signal generator 407 to generate location information characterizing position and/or movement of the applicator 406 via its coupling to the first location signal generator 407. More specifically, the location information may characterize locations from where propagating wave energy has been delivered to a target location. To this end, the spatial referencing module 410 may track and/or record (or otherwise acquire and utilize) instances of relative position of the applicator 406 and/or instances of motion of the applicator 406.

For interacting with the first location signal generator 407 to generate the location information, the spatial referencing module 410 may include a spatial field sensor 411 (i.e., a second position-defining device). The spatial field sensor 411 is preferably positioned at a fixed remote location from the applicator 406 (and thus the first location signal generator 407). For example, spatial field sensor 411 may be attached to a body comprising the target location, may be attached to a support structure supporting the body, may be attached to a fixed structure of a room in which the body is located, or the like.

Jointly, via signaling therebetween, the first location signal generator 407 and the spatial field sensor 411 enable determination of movement of the applicator 406 and/or position of the applicator 406 (i.e., tracking functionality). For example, the spatial field sensor 411 may comprise a spatial field sensor or other type of spatial location referencing device that enables location of the first location signal generator 407 (e.g., a spatial field generator) and thus the applicator 406 to be determined with sufficient resolution for enabling the location information to be generated (preferably in a real-time manner) as a function of location of the applicator 406. In some embodiments, a source of emission of propagating waves from the applicator 406 may be all or part of the first location signal generator 407 (e.g., point source of the propagating wave energy defines the instant location of the applicator 406).

The energy delivery management module 415 includes an information processing apparatus 416. The energy delivery management module 415 is operably coupled to the treatment energy delivery module 405, the spatial referencing module 410, and the imaging module 420. Through such coupling, the information processing apparatus 416 may receive information from and/or provide information to the modules coupled to the energy delivery management module 415. Via such information communication, the information processing apparatus 416 is operable to receive spatial position information and energy delivery information outputted by the treatment energy delivery module 405 and is operable for receiving image information and spatial position information outputted by a transducer 421 of the imaging energy delivery module 420.

Energy delivery information outputted by the treatment energy delivery module 405 characterizes delivery of propagating wave energy to a target location. In some embodiments, the energy delivery management module 415 may generate energy delivery information that characterizes propagating wave energy delivered to the target location. The energy delivery management module 415 may be operably coupled to the treatment energy delivery module 405 in a plurality of ways, including, but not limited to: unitarily embodied with the treatment energy delivery module 405, integral with the treatment energy delivery module 405, and a discrete unit operably connected to the treatment energy delivery module 405.

Generating and/or managing energy delivery information may include accessing recorded energy delivery information corresponding to a first portion of a session of propagating wave energy treatment and determining energy delivery information during a second portion of the session of propagating wave energy treatment utilizing the energy delivery information from the first portion of the session. Examples of such energy delivery information include delivery location, energy amount, and duration of delivery.

The energy delivery management module 415 may be operable for creating a mapping of delivery of propagating wave energy relative to locations from where the propagating wave energy has been delivered to a target location and/or where propagating wave energy needs to be delivered to the target location. Such mapping may include correlation between two or more of the following parameters: one or more locations (e.g., area) of the target location subjected to initial delivery of propagating wave energy; one or more locations of the target location not subjected to initial delivery of propagating wave energy; one or more locations of the target location subjected to additional delivery of propagating wave energy; one or more locations of the target location requiring additional delivery of propagating wave energy; an amount of propagating wave energy delivered/required to be delivered; a duration of time propagating wave energy has been/is required to be delivered; an amount of propagating wave energy required to cause transformation from a baseline target location profile to the modified target location profile, and the like. In a preferred embodiment, the mapping correlates location of delivery to at least one of: one or more locations of the target location requiring initial delivery of propagating wave energy; one or more locations of the target location requiring additional delivery of propagating wave energy; an amount of propagating wave energy required to be delivered; and a duration of time propagating wave energy is required to be delivered.

In conjunction with mapping locations from where propagating wave energy has been delivered to the target location and determining one or more locations of the target location that require propagating wave energy treatment, the energy delivery management module 415 may be operable for determining a location of the target location where an amount of propagating wave energy has been delivered in an amount below a prescribed threshold. For example, to achieve a desired modified target location profile, a prescribed minimum amount of propagating wave energy must be delivered to all or a portion of the target location over all of or during each of one or more treatment sessions. In creating the mapping, the energy delivery management module 415 may associate locations from where propagating wave energy has been delivered to the target location to a location on a visual depiction (e.g., image, photograph, map, diagram, rendering, or the like) depicting at least a portion of the target location. Accordingly, the energy delivery management module 415 may be operable for assessing propagating wave energy delivered to locations of the target location, identifying one or more of such locations where the propagating wave energy delivered thereto is below a prescribed threshold, and causing one or more of such locations to be displayed on a visual depiction showing at least a portion of the target location. Thus, in one or more embodiments, the energy delivery management module 415 may be operable for receiving location information from a spatial referencing module defining a plurality of target locations within the target location, spatially mapping the target locations relative to each other to create a visual depiction of the target locations, and causing the visual depiction to be displayed in conjunction with an image depicting at least a portion of the target location.

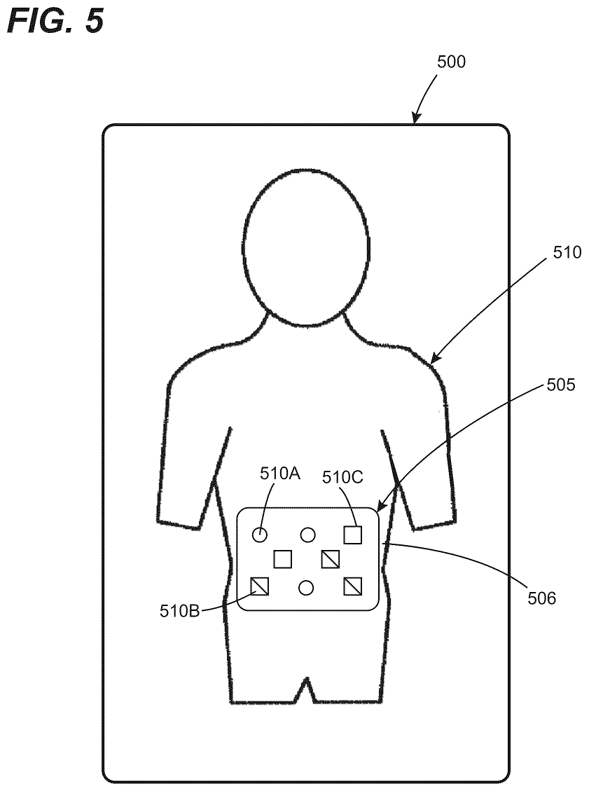

An example of a visual depiction 500 including an energy delivery mapping in accordance with one or more embodiments of the disclosures made herein is shown in FIG. 5. The visual depiction 500 includes a mapping 505 of categories of propagating wave energy delivery locations 510A, 510B, 510C relative to an abdominal region 506 (e.g., gut biome region treatment) of a patient 510. The propagating wave energy delivery location 510A is a delivery location category corresponding to not yet having had an initial delivery of propagating wave energy. The propagating wave energy delivery location 510B is a delivery location category corresponding to having had delivery of propagating wave energy (initial or otherwise) and not requiring additional delivery of propagating wave energy. The propagating wave energy delivery location 510C is a delivery location category corresponding to having had delivery of propagating wave energy (initial or otherwise) and requiring additional delivery of propagating wave energy. Other information that may be depicted on a location specific basis includes, but is not limited to, an amount (quantitatively or qualitatively depicted) of propagating wave energy delivered/required to be delivered; a duration of time propagating wave energy has been/is required to be delivered; and an amount of propagating wave energy required to cause transformation from the baseline target location biological profile to the modified target location biological profile.

The imaging module 420 may be a conventional ultrasound system or configured similarly to a conventional ultrasound system. A primary utility of a conventional ultrasound system is imaging of internal bodily structures. As is well known, a conventional ultrasound system includes an emitter that emits ultrasonic energy waves and a sensor that senses a reflected portion of such emitted ultrasonic energy waves. The emitter and sensor are jointly referred to herein as a transducer. The reflected portion of such emitted ultrasonic energy waves are processed to derive an image of the object (e.g., internal bodily structure) from which the reflected portion of such emitted ultrasonic energy waves are reflected. The image may be visually displayed, recorded as data, and/or outputted as a signal defining such data.

In accordance with one or more embodiments of the disclosures made herein, a second location signal generator 422 may be coupled to (e.g., fixedly attached to) the transducer 421 for outputting a signal that enables relative position and/or motion of the transducer 421 to be tracked and/or recorded. The spatial referencing module 410 is operable to utilize a spatial position signal outputted by the second location signal generator 422 to generate location information characterizing position and/or movement of the transducer 421 via its coupling to the second location signal generator 422. Operability of the spatial referencing module 410 is discussed above. The location information for the transducer 421 may be used to localize (e.g., characterize location of) and/or visualize a target bodily structure associated with a biome region in real time. For example, the transducer 421 in combination with the second location signal generator 422 may be used to generate an image and associated relative reference locations thereof of the target bodily structure. The energy delivery management module 415 may receive a signal from the imaging module 420 processible to facilitate mapping of energy delivery information as a function of, for example, such image and associated relative reference locations thereof of the target bodily structure generated through use of the transducer 421 and second location signal generator 422 coupled thereto.

The information processing apparatus 416 may provide for various spatial reference point transformations. In some embodiments of such a transformation, a known reference point of a spatial field generator may be computationally translated from a physical location to a virtual location. For example, as shown in FIG. 1B, the spatial location SL110 of the applicator 110 (e.g., physical or magnetic location defined by the spatial field generator 111) may be computationally translated to a virtual reference location VRL110 of the applicator 110. Here, the spatial location SL110 and the virtual location VRL110 are both located on the energy emissions axis EA110 of the applicator 110 and the virtual location VRL110 is located on a body contact surface CS110 of the applicator 110.

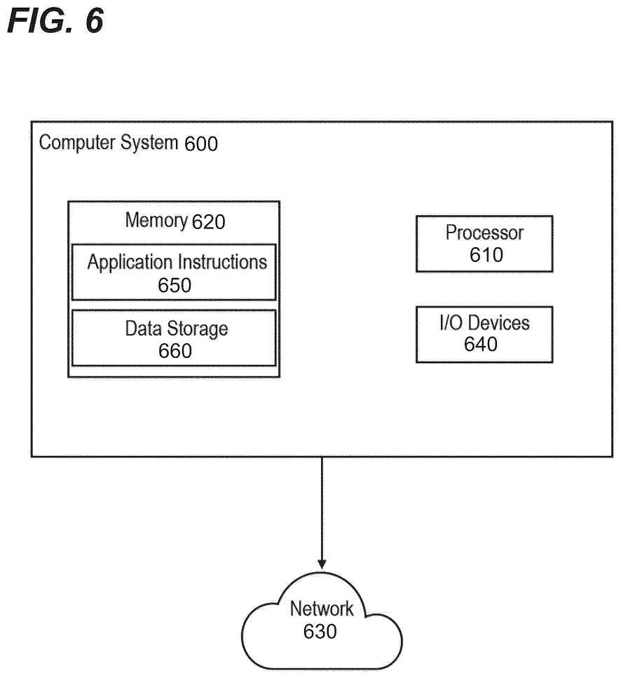

Referring to FIG. 6, a computing system 600 configured in accordance with one or more embodiments of the disclosures made herein is shown. The computing system 600 may be configured to perform functionalities embodied by information processing apparatuses and/or feedback modules in accordance with embodiments of the disclosures made herein. Examples of such functionality are discussed above in reference to the method of FIGS. 1-4 and the location delivery mapping of FIG. 5. The computing systems may thus be embodied as a special-purpose computing configured for carrying out processor-implemented methodologies to automate various algorithmic operations (e.g., step-wise instruction, computer program code, or the like) embodying such methodologies. The computing apparatus 600 may be a standalone apparatus or may be fully or partially integrated with the treatment energy delivery module 405. The computing system 600 is an example of an information processing apparatus.

The computer system 600 may include one or more processors (processor(s) 610) coupled to a memory 620 via an input/output (I/O) interface. Computer system 600 may further include a network interface to communicate with a network 630. The computer system 600 includes one or more input/output (I/O) devices 640, such as video device(s) (e.g., a camera), audio device(s), and visual display(s). In some embodiments, similar I/O devices 640 may be separate from computer system 600 and may interact with one or more nodes of the computer system 600 through a wired or wireless connection, such as over a network interface.

Processor(s) 610 suitable for the execution of a computer program include both general and special purpose microprocessors and any one or more processors of any digital computing device. Processor(s) 610 will receive instructions and data from a read-only memory or a random-access memory or both. The essential elements of a computing device are a processor for performing actions in accordance with instructions and one or more memory devices for storing instructions and data. Generally, a computing device will also include, or be operatively coupled to receive data from or transfer data to, or both, one or more mass storage devices for storing data, e.g., magnetic, magneto-optical disks, or optical disks; however, a computing device need not have such devices. Moreover, a computing device can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

A network interface may be configured to allow data to be exchanged between the computer system 600 and other devices attached to a network 630, such as other computer systems, or between nodes of the computer system 600, or other system modules in accordance with embodiments of the disclosures made herein. In various embodiments, the network interface may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol.

Memory 620 may include application instructions 650, configured to implement certain aspects of embodiments described herein (e.g., functionality for modifying biome biological profile of a patient to mitigate a health disorder), and data storage (e.g., comprising or consisting of a database) 660, comprising various data accessible by the application instructions 650. In one embodiment, the application instructions 650 may include software elements (set of processor-executable instructions embodied as computer program code) corresponding to one or more of the various embodiments described herein. For example, application instructions 650 may be implemented in various embodiments using any desired programming language, scripting language, or combination of programming languages and/or scripting languages (e.g., C, C++, C#, JAVA®, JAVASCRIPT®, PERL®, etc.).

Memory 620 may include information associated with implementing functionality for modifying biome biological profile of a patient to mitigate a health disorder in accordance with one or more embodiments of the disclosures made herein. For example, memory 620 may be non-transient computer-readable media having information stored thereon embodying functionality provided by the energy delivery management module 415 and spatial referencing module 410 discussed above in reference to FIG. 4.

The operations and actions of the computer system 600 described in connection with the embodiments disclosed herein may be embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to processor(s) 610 such that processor(s) 610 can read information from, and write information to, the storage medium. In the alternative, the storage medium may be integrated into processor(s) 610. Further, in some embodiments, processor(s) 610 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

Also, any connection may be associated with a computer-readable medium and, more preferably, a non-transitory computer-readable medium/media. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

The term “computer-readable medium” as used herein refers to any medium (but, preferably non-transitory) that participates in providing instructions to processor(s) 610 for execution. Such a medium may take many forms, including but not limited to, non-volatile media, volatile media, and transmission media. Non-volatile media includes, for example, optical or magnetic disks. Volatile media includes dynamic memory, such as memory 620. Transmission media includes coaxial cables, copper wire, and fiber optics, including the wires that comprise a bus. Transmission media can also take the form of acoustic or light waves, such as those generated during radio-wave and infra-red data communications. Common forms of computer-readable media include, for example, a floppy disk, a flexible disk, hard disk, magnetic tape, or any other magnetic medium, a CD-ROM, any other optical medium, punchcards, papertape, any other physical medium with patterns of holes, a RAM, a PROM, and EPROM, a FLASH-EPROM, any other memory chip or cartridge, a carrier wave as described hereinafter, or any other medium from which a computer can read. Various forms of computer readable media may be involved in carrying one or more sequences of one or more instructions to processor(s) 610 for execution. For example, the instructions may initially be carried on a magnetic disk of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on a bus. A bus in this regard may carry data to memory (e.g., memory 620), from which a processor (e.g., the one or more processors 610) retrieves and executes the instructions. The instructions received by memory may optionally be stored on a storage device either before or after execution by a processor. To this end, the one or more processors 610, the memory 620 and I/O devices 640 are preferably interconnected with each other via a bus or similar communication structure.

In some embodiments, the computing system 600 is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In preferred embodiments, treatment of tissue with propagating wave energy in accordance with one or more embodiments of the disclosures made herein may activate and/or normalize cellular function. To this end, such treatment may include activating an acoustic shock wave source (e.g., generator) to emit acoustic shock waves and subjecting target tissue of a patient to one or more treatments of exposure to acoustic shock waves. The acoustic shock waves provide pressure pulses each comprising a plurality of cycles of a positive pressure part and a negative pressure part. Subjecting the target tissue of the patient to the plurality of treatments of exposure to the acoustic shock waves is performed for causing stimulation of cells of the target tissue to initiate genetic expression of cells of the target tissue. Such stimulation of cells of the target tissue to initiate genetic expression may cause one or more of release of exosomes from cells of targeted (i.e., treated) tissue, activation of at least one cellular receptor, shedding of micro-vesicles from the cells of targeted tissue, and release of at least one of a protein, cytokines, and mRNA from the cells of targeted tissue into the extracellular matrix of the targeted tissue.

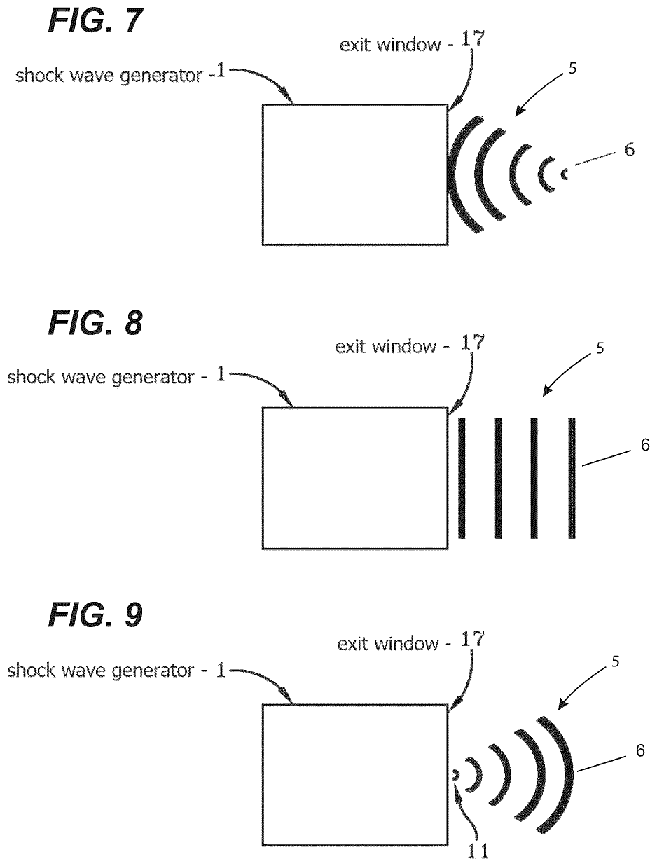

With reference to FIGS. 7-9, a variety of schematic views of acoustic shock waves or pressure pulses are described. The following description of the proper amplitude and pressure pulse intensities of the shock waves are provided along with a description of how the shock waves actually function. For the purpose of describing, the shock waves were used as exemplary and are intended to include all of the wave patterns discussed in the figures as possible treatment patterns. Specific details of acoustic shock waves and treatment therewith are disclosed in U.S. Pat. Nos. 7,470,240; 7,507,213; 7,601,127; 7,695,443; 8,257,282; 11,311,454; 11,389,371, which are incorporated herein in their entirety by reference.

FIG. 7 is a simplified depiction of a pressure pulse/shock wave (PP/SW) generator, such as a shock wave head, showing focusing characteristics of transmitted acoustic pressure pulses 5. Numeral 1 indicates the position of a pressure pulse generator according to the present invention (i.e., a propagating energy wave source which may be embodied as a treatment energy delivery module), which generates pressure pulses 5 and, via a focusing element, focuses it outside the housing to treat diseases. The affected tissue or organ is generally located in or near the focal point which is located in or near position 6. At position 17 a water cushion or any other kind of exit window for the acoustical energy is located.

FIG. 8 is a simplified depiction of a pressure pulse/shock wave generator, such as a shock wave head, with plane wave characteristics. Numeral 1 indicates the position of a pressure pulse generator according to the present invention (i.e., a propagating energy wave source which may be embodied as a treatment energy delivery module), which generates pressure pulses 5 which are leaving the housing at the position 17, which may be a water cushion or any other kind of exit window. Somewhat even, also referred to herein as “disturbed,” wave characteristics can be generated, in case a paraboloid is used as a reflecting element, with a point source (e.g. electrode) that is located in the focal point of the paraboloid. The pressure pulses 5 will be transmitted into the patient's body via a coupling media such as, e.g., ultrasound gel or oil and their amplitudes will be attenuated with increasing distance from the exit window 17.

FIG. 9 is a simplified depiction of a pressure pulse shock wave generator (i.e., a shock wave head of a propagating energy wave source) with divergent wave characteristics. Numeral 1 indicates the position of a pressure pulse generator according to the present invention (i.e., a propagating energy wave source which may be embodied as a treatment energy delivery module), which generates pressure pulses 5 which are leaving the housing at the position 17, which may be a water cushion or any other kind of exit window. The divergent wave fronts may be leaving the exit window 17 at point 11 where the amplitude of the wave front is high. This point 11 could be regarded as the source point for pressure pulses 5. The pressure pulse source may be a point source, that is, pressure pulses 5 may be generated by an electrical discharge of an electrode under water between electrode tips. However, pressure pulses 5 may also be generated, for example, by an explosion, referred to as a ballistic pressure pulse. The divergent characteristics of the wave front may be a consequence of the mechanical setup.

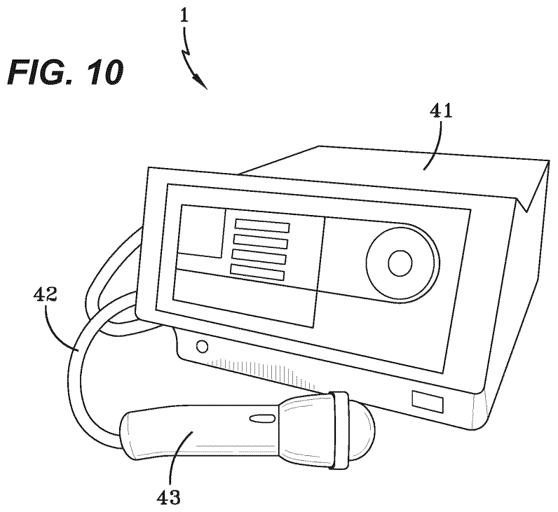

With reference to FIG. 10, an exemplary a pulse generator 1 (i.e., a propagating energy wave source which may be embodied as a treatment energy delivery module) is shown. The shock wave apparatus has a generator 41 connected by a flexible hose 42 with fluid conduits extending from the shock wave generator 41 to an applicator 43 which transmits the acoustic waves when coupled to the skin by using a fluid or acoustic gel. The applicator 43 as illustrated has a body that enables a technician to hold the applicator 43 and as illustrated this applicator is an electrohydraulic device that is filled with fluid to facilitate the transmission of the shock waves. The fluid may expand a flexible membrane in such a fashion that the membrane extends outwardly in a balloon shape fashion. As shown, this type of applicator 43 has a hydraulic spark generator using either focused or unfocused shock waves, preferably in a low energy level, less than the range of 0.01 mJ/mm² to 0.5 mJ/mm². In some embodiments, focused or unfocused shock waves may have an energy level up to at least 100.0 mJ/mm². The flexible hose 42 is connected to a fluid supply that fills the applicator 43 and expands the flexible membrane when filled. Alternatively, a ballistic, piezoelectric, or spherical acoustic shock wave device can be used to generate the desired waves.

Many different embodiments have been disclosed herein, in connection with the above description and the drawings. It will be understood that it would be unduly repetitious and obfuscating to describe and illustrate every combination and sub-combination of these embodiments. Accordingly, all embodiments can be combined in any way and/or combination, and the present specification, including the drawings, shall be construed to constitute a complete written description of all combinations and sub-combinations of the embodiments described herein, and of the manner and process of making and using them, and shall support claims to any such combination or sub-combination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a sub-combination or variation of a sub-combination.

Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the invention in all its aspects. Although the invention has been described with reference to particular means, materials and embodiments, the invention is not intended to be limited to the particulars disclosed; rather, the invention extends to all functionally equivalent technologies, structures, methods and uses such as are within the scope of the appended claims.

What is claimed is:

1. A system for treating a patient with acoustic shock waves, comprising:
an ultrasound transducer configured to output transducer spatial position information, to emit ultrasound energy for imaging a target location within a body of the patient, and to output imaging information generated by said imaging;
an acoustic shock wave applicator configured to emit acoustic shock waves and to output applicator spatial position information; and
an information processing apparatus coupled to the ultrasound transducer and to the acoustic shock wave applicator for receiving said transducer spatial position information, said applicator spatial position information, and said imaging information respectively therefrom, wherein the information processing apparatus is configured for:
generating a mapping function using said transducer spatial position information, said applicator spatial position information, and said imaging information, wherein the mapping function correlates a plurality of transducer spatial positions derived from said transducer spatial position information to a respective portion of said imaging information and wherein the respective portion of the imaging information was generated with the ultrasound transducer in a particular one of said transducer spatial positions.

2. The system of claim 1, wherein the ultrasound transducer and the acoustic shock wave applicator each comprise a first portion of a spatial position sensing apparatus that respectively outputs a signal representative of said spatial position information.

3. The system of claim 2, wherein the first portion of the spatial position sensing apparatus includes a spatial field generator.

4. The system of claim 3, wherein the spatial field generator includes a magnetic field generator.

5. The system of claim 2, further comprising:
a second portion of the spatial position sensing apparatus is one of coupled to and integral with the information processing apparatus, wherein the second portion of the spatial position sensing apparatus receives said spatial position information and determines therefrom a relative spatial position with respect to a fixed reference position.

6. The system of claim 5, wherein:
the first portion of the spatial position sensing apparatus includes a spatial field generator; and
the second portion of the spatial position sensing apparatus includes a spatial field sensor.

7. The system of claim 1, further comprising:
a spatial referencing module is one of coupled to and integral with the information processing apparatus, wherein the spatial referencing module receives said spatial position information from the ultrasound transducer and from the acoustic shock wave applicator and determines therefrom a respective spatial position thereof with respect to a fixed reference position.

8. The system of claim 1, wherein the respective portion of said imaging information enables an image of the target location to be rendered therefrom.

9. The system of claim 1, wherein the information processing apparatus is further configured for:
determining, as a function of the mapping function and a particular one of a plurality of applicator spatial positions, the respective portion of said imaging information generated with the ultrasound transducer in the particular one of said transducer spatial positions that is same or sufficiently the same as the particular one of said applicator spatial positions thereby enabling real-time spatial position of the acoustic shock wave applicator to be adjusted based at least partially upon said imaging information.

10. The system of claim 9, wherein the information processing apparatus is further configured for:
deriving each of said applicator spatial positions from said applicator spatial position information.

11. The system of claim 9, wherein the information processing apparatus is further configured for at least one of:
causing display of the respective portion of said imaging information in real-time as a function of said applicator spatial position information; and
providing confirmation that an energy wave emissions axis of the acoustic shock wave applicator is colinearly aligned with or sufficiently colinearly aligned with an energy wave emissions axis of the ultrasound transducer when the respective portion of said imaging information was generated.

12. The system of claim 9, wherein being in the particular one of said transducer spatial positions that is same or sufficiently the same as the particular one of said applicator spatial positions includes an energy wave emissions axis of the acoustic shock wave applicator being colinearly aligned with or sufficiently colinearly aligned with an energy wave emissions axis of the ultrasound transducer when the respective portion of said imaging information was generated.

13. The system of claim 12, wherein the ultrasound transducer and the acoustic shock wave applicator each comprise a first portion of a spatial position sensing apparatus that respectively outputs a signal representative of said spatial position information.

14. The system of claim 13, wherein the first portion of the spatial position sensing apparatus includes a spatial field generator.

15. The system of claim 14, wherein the spatial field generator includes a magnetic field generator.

16. The system of claim 13, further comprising:
a second portion of the spatial position sensing apparatus is one of coupled to and integral with the information processing apparatus, wherein the second portion of the spatial position sensing apparatus receives said spatial position information and determines therefrom a relative spatial position with respect to a fixed reference position.

17. The system of claim 16, wherein:
the first portion of the spatial position sensing apparatus includes a spatial field generator; and
the second portion of the spatial position sensing apparatus includes a spatial field sensor.

18. The system of claim 9, further comprising:
a spatial referencing module is one of coupled to and integral with the information processing apparatus, wherein the spatial referencing module receives said spatial position information from the ultrasound transducer and from the acoustic shock wave applicator and determines therefrom a respective spatial position thereof with respect to a fixed reference position.

19. An apparatus for treating a patient using propagating wave energy, comprising:
a transducer operable to emit a first configuration of propagating wave energy for enabling images of a target location within a body of the patient to be generated, wherein each of the images corresponds to a respective one of a plurality of transducer spatial positions relative to the target location;
an applicator operable to emit a second configuration of propagating wave energy from a plurality of applicator spatial positions relative to the target location for treating a wellness condition at the target location; and
a feedback module operably coupled to the transducer and to the applicator, wherein the feedback module correlates a current one of said applicator spatial positions to a particular one of said images when the current one of said applicator spatial positions is the same as or sufficiently similar to the respective one of said transducer spatial positions in which the transducer was positioned for enabling the particular one of said images to be generated.

20. The apparatus of claim 19, wherein:
the first configuration of propagating wave energy is ultrasound energy; and
the second configuration of propagating wave energy is acoustic shock wave energy.

21. The apparatus of claim 19, wherein:
the first configuration of propagating wave energy is selected for enabling imaging of the target location; and
the second configuration of propagating wave energy is selected for treating the wellness condition.

22. The apparatus of claim 19, wherein the transducer and the applicator each include a spatial field generator integral therewith.

23. The apparatus of claim 22, wherein the spatial field generator is a magnetic spatial field generator.

24. The apparatus of claim 22, wherein each spatial field generator has a longitudinal reference axis defined thereby aligned with an energy emissions axis of the respective one of the transducer and the applicator.

25. The apparatus of claim 24, wherein the spatial field generator is a magnetic spatial field generator.

26. The apparatus of claim 19, further comprising:
a spatial referencing module coupled to the feedback module, wherein the spatial referencing module receives first spatial position information from the ultrasound transducer and second spatial position information from the acoustic shock wave applicator and determines therefrom a respective spatial position thereof with respect to a fixed reference position.

27. The apparatus of claim 19, wherein:
the spatial referencing module includes a spatial field sensor; and
the transducer and the applicator each include a spatial field generator integral therewith.

28. The apparatus of claim 27, wherein the spatial field generator is a magnetic spatial field generator.

29. The apparatus of claim 27, wherein each spatial field generator has a longitudinal reference axis defined thereby aligned with an energy emissions axis of the respective one of the transducer and the applicator.

30. The apparatus of claim 29, wherein the spatial field generator is a magnetic spatial field generator.

31. The apparatus of claim 19, wherein the current one of said applicator spatial positions being sufficiently similar to the respective one of said transducer spatial positions includes at least one of:
an energy emissions axis of the applicator when in the current one of said applicator spatial positions being within a prescribed lateral offset from an energy emissions axis of the transducer when in the respective one of said transducer spatial positions; and
an energy emissions axis of the applicator when in the current one of said applicator spatial positions being within a prescribed angular offset from an energy emissions axis of the transducer when in the respective one of said transducer spatial positions.

32. The apparatus of claim 31, further comprising:
a spatial referencing module coupled to the feedback module, wherein the spatial referencing module receives first spatial position information from the ultrasound transducer and second spatial position information from the acoustic shock wave applicator and determines therefrom a respective spatial position thereof with respect to a fixed reference position.

33. The apparatus of claim 32, wherein:
the spatial referencing module includes a spatial field sensor; and
the transducer and the applicator each include a spatial field generator integral therewith.

34. The apparatus of claim 33, wherein each spatial field generator has a longitudinal reference axis defined thereby aligned with an energy emissions axis of the respective one of the transducer and the applicator.

35. A non-transitory computer-readable memory medium containing program instructions for controlling one or more data processors, when executed, to provide system-aided delivery of propagating wave energy for treating a wellness condition at a target location within a body of a patient, by performing a method comprising:
receiving first spatial position information defining spatial positions of a first handheld device relative to the body, wherein the first handheld device emits a first configuration of propagating wave energy;
receiving imaging information outputted by the first handheld device, wherein said imaging information is generated by the first handheld device using said propagating wave energy emitted thereby for imaging a target location within the body and wherein said imaging is performed with the first handheld device in a plurality of said spatial positions thereof;
receiving second spatial position information defining spatial positions of a second handheld device relative to the body, wherein the second handheld device emits a second configuration of propagating wave energy selected for treating the wellness condition at the target location; and
generating an image of the target location corresponding to a particular one of said spatial positions of the second handheld device, wherein said generating is performed as a function of said first spatial position information, said second spatial position information, and said imaging information.

36. The non-transitory computer-readable memory medium of claim 35, wherein:
the first handheld device is an ultrasound transducer; and
the second handheld device is an acoustic shock wave applicator.

37. The non-transitory computer-readable memory medium of claim 35, wherein:
the first configuration of propagating wave energy is an ultrasound energy; and
the second configuration of propagating wave energy is an acoustic shock wave energy.

38. The non-transitory computer-readable memory medium of claim 35, wherein:
the first handheld device and the second handheld device each include a spatial field generator integral therewith; and
receiving said first spatial position information and receiving said second spatial position information respectively include receiving information characterizing an orthogonal position of a spatial field generator of the respective one of said handheld devices relative to a reference location and information characterizing angular rotation of the spatial field generator relative to a reference axis.

39. The non-transitory computer-readable memory medium of claim 35, wherein generating the image of the target location corresponding to the particular one of said spatial positions of the second handheld device includes:
accessing a mapping function correlating a plurality of first handheld device spatial positions derived from said first handheld device spatial position information to a respective portion of said imaging information and wherein the respective portion of the imaging information was generated with the first handheld device in a particular one of said transducer spatial positions; and
determining, as a function of the mapping function and the particular one of said spatial positions of the second handheld device, the respective portion of said imaging information generated with the first handheld device in the particular one of said first handheld device spatial positions that is same or sufficiently the same as the particular one of said spatial positions of the second handheld device.

40. The non-transitory computer-readable memory medium of claim 39, wherein the information processing apparatus is further configured for at least one of:
causing display of the respective portion of said imaging information in real-time as a function of said second handheld device spatial position information; and
providing confirmation that an energy wave emissions axis of the second handheld device is colinearly aligned with or sufficiently colinearly aligned with an energy wave emissions axis of the first handheld device when the respective portion of said imaging information was generated.

41. The non-transitory computer-readable memory medium of claim 39, wherein being in the particular one of said first handheld device spatial positions that is same or sufficiently the same as the particular one of said second handheld device spatial positions includes an energy wave emissions axis of the second handheld device being colinearly aligned with or sufficiently colinearly aligned with an energy wave emissions axis of the first handheld device when the respective portion of said imaging information was generated.

42. The non-transitory computer-readable memory medium of claim 39, wherein:
the first handheld device and the second handheld device each include a spatial field generator integral therewith; and
receiving said first spatial position information and receiving said second spatial position information respectively include receiving information characterizing an orthogonal position of a spatial field generator of the respective one of said handheld devices relative to a reference location and information characterizing angular rotation of the spatial field generator relative to a reference axis.

43. The non-transitory computer-readable memory medium of claim 35, wherein generating the image of the target location corresponding to the particular one of said spatial positions of the second handheld device includes:
determining, as a function of the particular one of said spatial positions of the second handheld device, a respective portion of said imaging information generated with the first handheld device in a particular one of said first handheld device spatial positions that is same or sufficiently the same as the particular one of said spatial positions of the second handheld device and wherein the respective portion of the imaging information was generated with the first handheld device in the particular one of said first handheld device spatial positions.

44. The non-transitory computer-readable memory medium of claim 43, wherein the information processing apparatus is further configured for at least one of:
causing display of the respective portion of said imaging information in real-time as a function of said second handheld device spatial position information; and
providing confirmation that an energy wave emissions axis of the second handheld device is colinearly aligned with or sufficiently colinearly aligned with an energy wave emissions axis of the first handheld device when the respective portion of said imaging information was generated.

45. The non-transitory computer-readable memory medium of claim 44, wherein being in the particular one of said first handheld device spatial positions that is same or sufficiently the same as the particular one of said second handheld device spatial positions includes an energy wave emissions axis of the second handheld device being colinearly aligned with or sufficiently colinearly aligned with an energy wave emissions axis of the first handheld device when the respective portion of said imaging information was generated.

46. The non-transitory computer-readable memory medium of claim 44, wherein:
the first handheld device and the second handheld device each include a spatial field generator integral therewith; and
receiving said first spatial position information and receiving said second spatial position information respectively include receiving information characterizing an orthogonal position of a spatial field generator of the respective one of said handheld devices relative to a reference location and information characterizing angular rotation of the spatial field generator relative to a reference axis.

47. The non-transitory computer-readable memory medium of claim 35, wherein the information processing apparatus is further configured for at least one of:
causing display of the respective portion of said imaging information in real-time as a function of said second handheld device spatial position information; and
providing confirmation that an energy wave emissions axis of the second handheld device is colinearly aligned with or sufficiently colinearly aligned with an energy wave emissions axis of the first handheld device when the respective portion of said imaging information was generated.

48. The non-transitory computer-readable memory medium of claim 47, wherein being in the particular one of said first handheld device spatial positions that is same or sufficiently the same as the particular one of said second handheld device spatial positions includes an energy wave emissions axis of the second handheld device being colinearly aligned with or sufficiently colinearly aligned with an energy wave emissions axis of the first handheld device when the respective portion of said imaging information was generated.

49. The non-transitory computer-readable memory medium of claim 48, wherein:
the first handheld device and the second handheld device each include a spatial field generator integral therewith; and
receiving said first spatial position information and receiving said second spatial position information respectively includes receiving information characterizing an orthogonal position of a spatial field generator of the respective one of said handheld devices relative to a reference location and information characterizing angular rotation of the spatial field generator relative to a reference axis.

50. The non-transitory computer-readable memory medium of claim 35, wherein being in the particular one of said first handheld device spatial positions that is same or sufficiently the same as the particular one of said second handheld device spatial positions includes an energy wave emissions axis of the second handheld device being colinearly aligned with or sufficiently colinearly aligned with an energy wave emissions axis of the first handheld device when the respective portion of said imaging information was generated.
